(19) United States
(12) Patent Application Publication
(10) Pub. No.: US 2017/0154759 A1
(43) Pub. Date: Jun. 1, 2017

(54) MASS SPECTROMETRY IMAGING OF BENIGN MELANOCYTIC NEVI AND MALIGNANT MELANOMAS

(71) Applicants: Rossitza Zinovieva Lazova, Branford, CT (US); Erin Heather Seeley, Morgantown, WV (US)

(72) Inventors: Rossitza Zinovieva Lazova, Branford, CT (US); Erin Heather Seeley, Morgantown, WV (US)

(73) Assignees: Protea Biosciences, Inc., Morgantown, WV (US); Yale University, New Haven, CT (US)

(21) Appl. No.: 15/288,419

(22) Filed: Oct. 7, 2016

Related U.S. Application Data
(60) Provisional application No. 62/238,313, filed on Oct. 7, 2015.

(51) Int. Cl. H01J 49/00; H01J 49/16; G01N 33/483

(52) U.S. Cl. CPC H01J 49/0036; G01N 33/4833; H01J 49/164

(57) ABSTRACT

A method of differentiating benign melanocytic nevi from malignant melanomas is disclosed. The method generally includes subjecting a skin lesion sample from a patient to mass spectrometry to obtain a mass spectrometry protein profile. This profile is compared to mass spectrometry protein profiles of reference samples, which include known normal, benign melanocytic nevi, and/or malignant melanomas. Classification of the skin lesion sample as a benign melanocytic nevus or a malignant melanoma is based on similarities and difference between the mass spectrometry protein profiles.

### CROSS REFERENCE TO RELATED APPLICATIONS

The present application claims the benefit under 35 U.S.C. §119(e) of prior U.S. Provisional Application No. 62/238,313 filed Oct. 7, 2015, the entire content of which is hereby incorporated by reference.

### TECHNICAL FIELD OF THE INVENTION

The present invention relates to mass spectrometry imaging, and in particular, systems and methods of using mass spectrometry imaging to differentiate between benign melanocytic nevi and malignant melanomas.

### BACKGROUND OF THE INVENTION

Matrix-assisted laser desorption ionization (MALDI) mass spectrometry imaging (MSI) may be used to analyze metabolites, peptides and proteins, DNA segments, and lipids directly from tissue sections with spatial fidelity. MALDI MSI may be performed on either fresh frozen or formalin fixed, paraffin embedded tissue specimens. MSI may be used to elucidate molecular signatures of different tumor types and grades including brain, oral, lung, breast, gastric, pancreatic, renal, ovarian and prostate cancers. Conventional MSI may suffer from one or more of the following limitations: spatial resolution, mass accuracy, spectral resolution, sensitivity, robustness, reproducibility, requirements for sample preparation, and degree of technical difficulty. Accordingly, more efficient mass spectrometry imaging systems and methods of making and using the same are desirable.

### SUMMARY OF THE INVENTION

The present invention relates to a method of differentiating benign melanocytic nevi from malignant melanomas. The method may generally comprise subjecting a skin lesion sample from a patient to mass spectrometry; obtaining a mass spectrometric protein profile from the lesion sample; comparing the mass spectrometric protein profile to a known normal, benign melanocytic nevus profile, and/or malignant melanoma profile; and classifying the lesion as a benign melanocytic nevus or malignant melanoma based on similarities and differences between the protein profiles.

### BRIEF DESCRIPTION OF THE DRAWINGS

The various embodiments described herein may be better understood by considering the following description in conjunction with the accompanying drawings, wherein:

FIG. 1 shows a histology guided mass spectrometry profiling workflow according to various embodiments of the invention described herein;

FIG. 2 shows an imaging and statistical analysis according to various embodiments of the invention described herein;

FIG. 3 shows images of benign melanocytic nevus and malignant melanoma as defined according to various embodiments of the invention described herein;

FIG. 4 shows a proteomic analysis signature according to various embodiments of the invention described herein;

FIG. 5 shows a comparison of an average spectrum for benign melanocytic nevus (top) and malignant melanoma (bottom) obtained using mass spectrometry methods according to various embodiments of the invention described herein; and

FIG. 6 shows a gel view of the average spectra shown in FIG. 5.

### DETAILED DISCUSSION

Throughout this description and in the appended claims, use of the singular includes the plural and plural encompasses the singular, unless specifically stated otherwise. For example, although reference is made herein to “a” profile, “a” protein, “a” section, and “an” image, one or more of any of these components and/or any other components described herein can be used.

As generally used herein, the terms “including” and “having” mean “comprising”. The word “comprising” and forms of the word “comprising”, as used in this description and in the claims, does not limit the present invention to exclude any variants or additions.

As generally used herein, the term “about” refers to an acceptable degree of error for the quantity measured, given the nature or precision of the measurements. Typical exemplary degrees of error may be within 20%, 10%, or 5% of a given value or range of values. Alternatively, and particularly in biological systems, the terms “about” refers to values within an order of magnitude, potentially within 5-fold or 2-fold of a given value.

All numerical quantities stated herein are approximate unless stated otherwise. Accordingly, the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as precisely as possible.

Any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this disclosure is intended to include all lower numerical limitations subsumed therein and any minimum numerical limitation recited in this disclosure is intended to include all higher numerical limitations subsumed therein. Accordingly, Applicants reserve the right to amend this specification, including the claims, to expressly recite any sub-range subsumed within the ranges expressly recited herein.

In the following description, certain details are set forth in order to provide a better understanding of various embodiments of the invention which use mass spectrometry imaging for molecular diagnosis of a benign or malignant state of a cell, group of cells, lesion, tissue sample, or the like. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

This disclosure describes various features, aspects, and advantages of various embodiments of the invention. It is understood, however, that this disclosure embraces numerous alternative embodiments that may be accomplished by combining any of the various features, aspects, and advantages of the various embodiments described herein in any combination or sub-combination that one of ordinary skill in the art may find useful. Such combinations or sub-combinations are intended to be included within the scope of this specification. As such, the claims may be amended to recite any features or aspects expressly or inherently described in, or otherwise expressly or inherently supported by, the present disclosure. Further, Applicants reserve the right to amend the claims to affirmatively disclaim any features or aspects that may be present in the prior art. The various embodiments disclosed and described in this disclosure may comprise, consist of, or consist essentially of the features and aspects as variously described herein.

According to certain embodiments, more efficient and/or cost-effective mass spectrometry imaging systems and methods of making and using the same are described.

This disclosure describes various elements, features, aspects, and advantages of various embodiments of mass spectrometry imaging systems and methods. It is to be understood that certain descriptions of the disclosed embodiments have been simplified to illustrate only those elements, features and aspects that are relevant to a clear understanding of the disclosed embodiments, while eliminating, for purposes of clarity, other elements, features and aspects. Persons having ordinary skill in the art, upon considering the present description of the disclosed embodiments, will recognize that various combinations or sub-combinations of the disclosed embodiments and other elements, features, and/or aspects may be desirable in a particular implementation or application of the disclosed embodiments. However, because such other elements and/or features may be readily ascertained by persons having ordinary skill upon considering the present description of the disclosed embodiments, and are not necessary for a complete understanding of the disclosed embodiments, a description of such elements and/or features is not provided herein. As such, it is to be understood that the description set forth herein is merely exemplary and illustrative of the disclosed embodiments and is not intended to limit the scope of the invention as defined solely by the claims.

The present disclosure describes mass spectrometry imaging for molecular diagnosis of a benign or malignant state of a cell, group of cells, blood, lymph, skin lesion, tissue sample, or the like. In embodiments of the methods of the invention, the sample is obtained from a skin lesion, a primary tumor, a lymph node, or a local or distant metastasis.

As generally used herein, the term “primary tumor” refers to a malignant tumor (also referred to as cancer) at a first site (i.e., in a first organ or part of the body). In general, when an area of cancer cells at the originating site become clinically detectable, it is referred to as a primary tumor. Some cancer cells also acquire the ability to penetrate and infiltrate surrounding normal tissues in the local area, forming a new tumor. The newly formed “daughter” tumor in the adjacent site within the tissue is called a local metastasis while the formation of a new tumor in a non-adjacent site is called a distant metastasis.

Cancer cells and tumors may be characterized by having different proteins, and different protein expression levels, and/or different forms of proteins than exist in complementary benign cell or tissue samples. Frequently, proteins exist in a sample in a plurality of different forms characterized by detectably different masses. These forms can result from either or both of pre- and post-translational modification. Pre-translational modified forms include allelic variants, splice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation.

When detecting or measuring a protein in a sample, the ability to differentiate between these different forms depends upon the nature of the difference and the method used to detect or measure the difference. For example, an immunoassay using a monoclonal antibody will detect all forms of a protein containing the epitope and will not distinguish between them. In diagnostic assays, this inability to distinguish different forms of a protein diminishes the power of the assay. Further, only known biomarkers may be targeted. Thus, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

Mass spectrometry is a particularly powerful methodology to detect different forms of a protein because the different forms typically have different masses that can be resolved by the technique (mass spectrometry exploits the intrinsic properties of mass and charge). Accordingly, if one form of a protein is a superior biomarker for a disease over another form of the protein, mass spectrometry may be able to specifically detect and measure the useful form where traditional immunoassay fails to distinguish the forms and fails to specifically detect the useful biomarker.

As such, an embodiment of the present invention includes a method of differentiating benign cells or tissues from malignant cells or tissue using mass spectrometry analysis. Traditional quantitative mass spectrometry has used electrospray ionization (ESI) followed by tandem mass spectrometry (MS/MS), while newer quantitative methods are being developed using matrix assisted laser desorption/ionization (MALDI) followed by time of flight (TOF) mass spectrometry (MS).

In ESI tandem mass spectroscopy (ESI/MS/MS), simultaneous analysis of both precursor ions and product ions is possible, thereby monitoring a single precursor product reaction and producing a signal only when the desired precursor ion is present. Protein quantification has been achieved by quantifying tryptic peptides. Secondary ion mass spectroscopy (SIMS) uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge.

Laser desorption mass spectroscopy (LD-MS) involves the use of a pulsed laser, which induces desorption of sample material from a sample site effectively. This method may be used in conjunction with a mass spectrometer, and can be performed simultaneously with ionization if one uses the right laser radiation wavelength. When coupled with Time-of-Flight (TOF) measurement, LD-MS is referred to as LDLPMS (Laser Desorption Laser Photoionization Mass Spectroscopy). The LDLPMS method of analysis gives instantaneous volatilization (fragmentation) of the sample which permits rapid analysis without any wet extraction chemistry. Signal intensity, or peak height, is measured as a function of travel time. The applied voltage and charge of the particular ion determines the kinetic energy, and separation of fragments is due to the different sizes causing different velocities. Each ion mass will thus have a different flight-time to the detector.

MALDI-TOF mass spectroscopy can quantify intact proteins in biological tissue and fluids, and is thus applicable to direct analysis of biological tissues and single cell organisms with the aim of characterizing endogenous peptide and protein constituents. The sample is generally mixed with a matrix material which facilitates desorption and ionization of the sample. The signal intensity, or peak height is measured as a function of travel time, where separation of the fragments is due to the different sizes causing different velocities through the drift space and different times of flight to the detector.

Analysis of benign and malignant cells or tissue samples using mass spectrometry provides different protein profiles. Analysis of the protein profiles from known benign and known malignant samples may be used to generate proteomic signatures for each, which may be dependent on the type of tissue and the type or severity of the malignancy. These proteomic signatures may then be used to predict or classify an unknown sample.

Thus, the present invention provides a method of differentiating benign cells or tissues from malignant cells or tissue using mass spectrometry analysis, where the method generally comprises (a) subjecting a sample from a patient to mass spectrometry, (b) obtaining a mass spectrometric protein profile from the sample, (c) comparing the mass spectrometric protein profile to mass spectrometric protein profiles of reference samples, wherein reference samples include a known normal cell or tissue sample, and/or a known malignant cell or tissue sample, and (d) classifying the sample as benign or malignant based on similarities and differences between the protein profile of the sample and the reference sample(s).

The present invention further describes the use of mass spectrometry imaging methods to differentiate between benign melanocytic nevi and malignant melanomas. Benign melanocytic nevus (pl. nevi), also known as a mole, is the medical term for sharply circumscribed and chronic lesions of the skin, commonly named birthmarks or beauty marks. While Nevi are benign by definition, 25% of malignant melanomas arise from pre-existing nevi.

Skin cancer is the most common form of cancer. There are two major types of skin cancer, keratinocyte cancers (basal and squamous cell carcinomas) and melanoma (malignant melanocytes). Although melanoma accounts for only 3 percent of the skin cancers, it is responsible for greater than 75% of skin cancer deaths and is the seventh most common malignancy in the United States. The National Cancer Institute estimates that 73,870 new cases of melanoma will be diagnosed in the United States in 2015.

Among cells composing skin, melanin-pigment-producing cells are referred to as pigment cells or melanocytes. When these melanocytes become cancerous, a malignant melanoma is developed (i.e., skin lesion). Thus, as used herein, the term “malignant melanoma” may refer to a type of skin cancer involving melanocyte cells. The term “melanoma cells”, as used herein, may refer to melanocytes that have become cancerous. Melanocytes, including melanoma cells, are well known to the skilled person and can be easily identified in a sample due to their location in the stratum basal of the epidermis as well as via melanocyte-specific markers including, but not limited to, Melan-A, HMB45, Protein S100, DCT and TRP2.

TABLE I

| Lesion Depth | 5 year survival rate |
|---|---|
| In situ | 100% |
| <0.75 mm | 99% |
| 0.75 mm-1.49 mm | 95% |
| 1.5 mm-2.49 mm | 82-86% |
| 2.5 mm-3.99 mm | 67-76% |
| >4.0 mm | 54-61% |

In general, the most important prognostic variable in malignant melanoma is the depth of the lesion or tumor (see Table I). Malignant melanoma is staged according to the severity of the disease, into stage 0 to stage 4, measured as a combination of the depth of the lesion or tumor and spread beyond the originating lesion. For example, stage 0 refers to melanoma in situ with 99.9% survival, the melanoma lesion generally having a lesion thickness of less than 0.75 mm. Stage I/II refers to invasive melanoma with 85 to 99% survival, the melanoma lesions generally having a lesion thickness of less than 2 mm. Stage II refers to high risk melanoma with 40 to 85% survival, and can be further divided into T2b (1.00 to 2.00 mm primary tumor thickness, with ulceration), T3a (2.00 to 4.00 mm primary tumor thickness, without ulceration), T3b (2.00 to 4.00 mm primary tumor thickness, with ulceration), T4a (4.00 mm or greater primary tumor thickness without ulceration) and T4b (4.00 mm or greater primary tumor thickness with ulceration). Stage III refers to local metastasis with 25 to 60% survival, and is characterized by positive lymph nodes. Finally, stage IV refers to distant metastasis with only 9 to 15% survival.

The standard care for a nevus suspected to be a malignant melanoma is excisional biopsy with 1-3 mm margins and re-biopsy if the sample is inadequate for diagnosis. In the United States, 1 to 2 million biopsies are performed each year to diagnose melanoma. Of these, 25% cannot be definitively classified using routine histopathology (Am J Surg Pathol, 2009, 33:1146-56), often leading to a misdiagnosis. This high rate of misdiagnosis is problematic on many levels. The false positives lead to unnecessary costly medical interventions (e.g., CT, MRI, or PET scans; additional biopsy), while the false negatives mean increased likelihood of a future presentation with more severe disease and risk of death.

Further, there can be considerable disagreement among dermatologists in the diagnosis of melanocytic lesions. In a 1996 study, 8 pathologists reviewed the same 37 slides containing thin sections of tissue prepared from biopsied nevus: 13 of the slides had complete diagnostic agreement from the 8 pathologists, 10 slides had one discordant diagnoses, 6 slides had 2 or more discordant diagnoses, and 8 slides had 3 or more discordant diagnoses.

The presently disclosed invention provides improved methods for differentiating between benign melanocytic nevi and malignant melanomas. More specifically, the present invention includes a targeted approach in which only discrete areas within a tissue sample are analyzed. Histological staining may also be used to guide the acquisition of mass spectrometry images so that each area analyzed may be enriched for a single cell type. Such analysis may be more conducive to statistical analysis and classification algorithm generation. Further, such methods may provide a biological insight into the classification which is not attainable through standard histological techniques (i.e., disease outcome, improved diagnostics, treatment responses, etc.).

Archival formalin-fixed, paraffin embedded tissue samples from benign melanocytic nevi and primary cutaneous malignant melanomas were analyzed by mass spectrometry imaging to identify proteomic differences (i.e., define a proteomic signature). A teaching set comprising 25 nevi and 25 melanomas, was used to generate statistical correlation models using a machine-learning algorithm, such as, for example, a genetic algorithm, and/or a linear discriminant analysis.

A genetic algorithm is a machine-learning algorithm that is similar to classical genetics. Initially, peaks from the spectra may be formed into groups (individuals) and evaluated for how well they may differentiate between the phenotypes (benign and malignant). The ones that perform poorly may be discarded while the best may be bread to form a new generation of offspring. The offspring may be then evaluated for their robustness and the best move on to the next generation. Additionally, mutations may be introduced to the sets of peaks at a user-defined rate to increase “genetic” variability. A maximum number of generations is set that the genetic algorithm is allowed to run over, although, this is often not reached as the calculations are terminated when a local optimum is obtained.

For the genetic algorithm, a combination of peaks that separates best between benign nevi and malignant melanomas was established. A diagnosis of either nevus or melanoma was rendered on a separate validation set of 26 nevi and 29 melanomas based on the proteomic signature, which diagnosis was then correlated with the histopathologic diagnosis. Using the genetic algorithm, mass spectrometry imaging classified 28 of the 29 cases of malignant melanomas, and 22 of the 26 cases of benign melanocytic nevi correctly. The sensitivity for recognizing melanomas was 97% and the specificity was 85%.

A linear discriminant analysis is a machine-learning algorithm which may be used to dimensionally reduce a dataset onto a lower-dimensional space. In a linear discriminant analysis, the computer program is taught to recognize a total spectrum of peaks from the benign samples and the malignant samples. The total spectrum is used as a fingerprint for a diagnosis. Each new spectrum from a new sample is compared to these fingerprints and matched to the one to which it is more similar. By comparison, a genetic algorithm quantifies only a small subset of peaks from the spectrum that best differentiates the groups. All other peaks and parts of the spectra are ignored.

For the linear discriminant analysis, the teaching set comprising 25 nevi and 25 melanomas was used, and a 10% leave-out repeated random sub-sampling cross validation was employed to evaluate accuracy, which was found to be 100%. An optimized classifier was applied to a validation set of 25 nevi specimens and 29 melanoma specimens, where the mass spectrometry results were compared to the known patient diagnoses. The methods of the present invention showed a 100% validation accuracy for benign samples in the validation set, and 97% validation accuracy for malignant samples in the validation set.

The use of a linear discriminant analysis allows for an accurate differentiation between benign and malignant melanocytic lesions. Applying the classifier developed using the linear discriminant analysis resulted in a 100% classification accuracy, which was effective at diagnosing histologically difficult cases. Further, using traditional LC-MS/MS, several specific proteins were identified to be differentiated in the malignant melanocyte lesions (see Table 2).

TABLE 2

| | Wilcoxon | Area under | |
|---|---|---|---|
| m/z | p-value | ROC Curve | Putative ID |
| 1198.76 | 3.18E−12 | 0.9631 | Actin |
| 976.50 | 4.77E−09 | 0.8916 | Actin |
| 1489.90 | 2.78E−08 | 0.8673 | Trinucleotide repeat- |
| | | | containing gene 6C protein |
| 1399.78 | 2.78E−08 | 0.8679 | Tropomyosin |
| 1501.80 | 3.80E−08 | 0.8679 | Myosin |
| 1646.88 | 6.35E−08 | 0.8562 | Vitronectin |
| 1072.07 | 1.11E−07 | 0.8465 | Collagen alpha-1 |
| 1254.69 | 1.64E−07 | 0.8405 | Vimentin |

m/z - mass/charge ratio;
ROC is receiver operating characteristic

Thus, mass spectrometry imaging may be used to differentiate between benign melanocytic nevi and malignant melanomas in formalin-fixed, paraffin-embedded tissue sections based on proteomic differences. Mass spectrometry imaging may be an objective and reliable method that may be helpful in difficult cases, in which rendering a firm diagnosis of either benign nevus or malignant melanoma may be very difficult. The identification of protein expression profiles, which discriminate between benign melanocytic nevi and malignant melanomas, may lead to the discovery of clinically useful protein biomarkers and translate into tumor biomarkers that may be incorporated into standard diagnostic and treatment strategies.

In various embodiments, a method of differentiating benign melanocytic nevi from malignant melanomas may generally comprise (a) subjecting a skin lesion sample from a patient to mass spectrometry; (b) obtaining a mass spectrometry profile from the skin lesion sample; (c) comparing the mass spectrometry profile of the skin lesion sample to a reference profile, wherein the reference profile includes a statistical average profile from a plurality of known normal, benign melanocytic nevi, and/or a plurality of known malignant melanoma; and (d) classifying the skin lesion sample as a benign melanocytic nevus or a melanocytic malignant melanoma based on a level of similarity between the mass spectrometry profile of the skin lesion sample and the reference profile.

The method may have a sensitivity and a specificity of at least 95% in correctly classifying the skin lesion sample, such as at least 96%, at least 97%, at least 98%, at least 99%, or even at least 99.5%.

The method may comprise repeating the steps of (a)-(d) on the skin lesion, such as, for example, in 6-12 months, 6-18 months, 6-24 months, 12-18 months, 12-24 months or 18-24 months when the patient is identified as having benign melanocytic nevi.

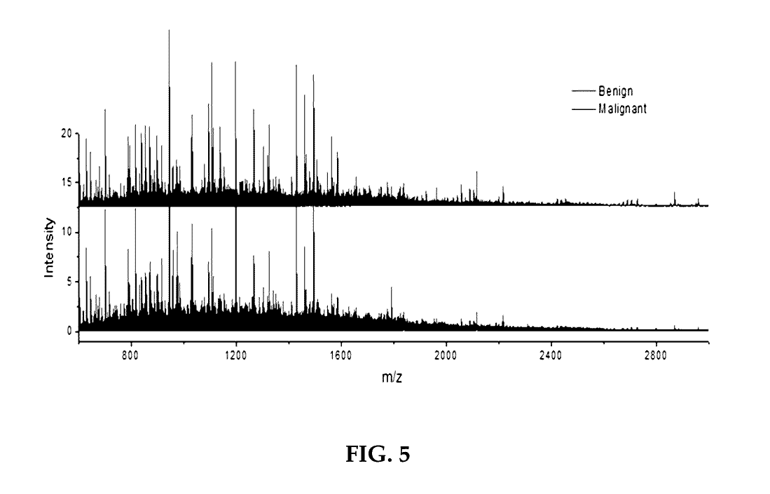

The classification may comprise use of a genetic algorithm or a linear discriminant analysis to generate a proteomic signature of a reference sample. The reference sample may include a proteomic signature of a known benign sample (i.e., cell, lesion, tumor) and a known malignant sample (i.e., cell, lesion, tumor). The mass spectrometric profile may comprise analysis of a specific subset of markers as defined by the genetic algorithm. The mass spectrometric profile may comprise an average spectrum from a known benign sample (FIG. 5, top) and an average spectrum from a known malignant sample (FIG. 5, bottom).

The mass spectrometry may comprise secondary ion mass spectrometry, laser desorption mass spectrometry, matrix assisted laser desorption mass spectrometry, electrospray mass spectrometry, desorption electrospray ionization, or laser ablation electrospray ionization mass spectrometry.

The method may comprise obtaining the sample from the patient.

The method may comprise making a treatment decision for the patient.

The method may comprise treating the patient with at least one of chemotherapy, immunotherapy, toxin therapy, surgery, and radiotherapy when the patient is identified as having malignant melanoma.

The method may comprise assessing one or more patient history parameters from the patient.

The method may comprise performing histologic analysis on the sample.

The method may comprise performing a mass spectrometric analysis of a known benign melanocytic nevus and/or melanocytic malignant melanoma lesion.

The method may comprise performing histologic analysis on the sample.

The method may comprise making a prediction of the patient's survival based on the classification.

The method may comprise determining a form of treatment, e.g., selecting a drug, based on the classification.

In various embodiments, the skin lesion sample comprises one of melanocytic components and stromal components. The melanocytic components may be confined to the area of the lesion. The method may comprise examining melanocytic and/or stromal components of the skin lesion sample. The method may comprise examining the normal (stromal) tissue directly adjacent and/or proximal to the skin lesion sample. The method may analyze the proteomic changes in the tissue microenvironment that may be indicative of a diagnosis.

The statistical average profile of the method may be generated using a genetic algorithm which defines a mass spectrometric profile comprising one or more markers.

In various embodiments, the mass spectrometric profile may comprises one or more markers. In various embodiments, the mass spectrometric profile may comprise one or more peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143.63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2. In various embodiments, the mass spectrometric profile may comprise one or more peptide peaks at m/z 976.50±0.2, m/z 1072.07±0.2, m/z 1138.61±0.2, m/z 1198.76±0.2, m/z 1254.69±0.2, m/z 1399.78±0.2, m/z 1489.90±0.2, m/z 1501.80±0.2, and m/z 1646.88±0.2.

The method may comprise using the mass spectrometric profile to determine which form of treatment may be more therapeutically effective for the patient. In various embodiments, the form of treatment may comprise a drug compound dosage regimen, antibody-drug conjugate dosage regimen, radiochemical compound dosage regimen, radiation therapy, and/or surgical excision,

The method may comprise immunohistochemical analysis on the skin lesion sample. In various embodiments, the patient may previously have had immunohistochemical analysis of the lesion. In various embodiments, the previous immunohistochemical analysis indicated that the lesion was a benign melanocytic nevus. In various embodiments, the previous immunohistochemical analysis indicated that the lesion was a malignant melanoma.

The statistical average profile of the method may be generated using a linear determinant analysis on the total spectrum from the plurality of known normal, benign melanocytic nevi, and/or the plurality of known malignant melanoma. The linear discriminant analysis may show a sensitivity and a specificity of at least 95% in correctly classifying the skin lesion sample.

The plurality of known normal, benign melanocytic nevi, and the plurality of known malignant melanoma may be classified by immunohistochemical analysis, genetic analysis, patient clinical outcome, or a combination thereof.

### Examples

The various embodiments herein may be better understood when read in conjunction with the following representative examples. The following examples are included for purposes of illustration and not limitation.

Tumor Specimens.

A total of 104 archival formalin-fixed, paraffin-embedded tissue samples from 51 histologically unequivocal benign melanocytic nevi (BMN) and 54 conventional primary cutaneous malignant melanomas (MM) were collected. All cases were re-reviewed by a dermatopathologist to confirm the diagnosis. The samples were randomly divided into 2 cohorts: a training set and a validation set. The training set consisted of 25 BMN and 25 MM. The validation set consisted of 26 BMN and 29 MM for the genetic algorithm or 25 BMN and 29 MM for the linear discriminant analysis.

Mass Spectrometry Analysis.

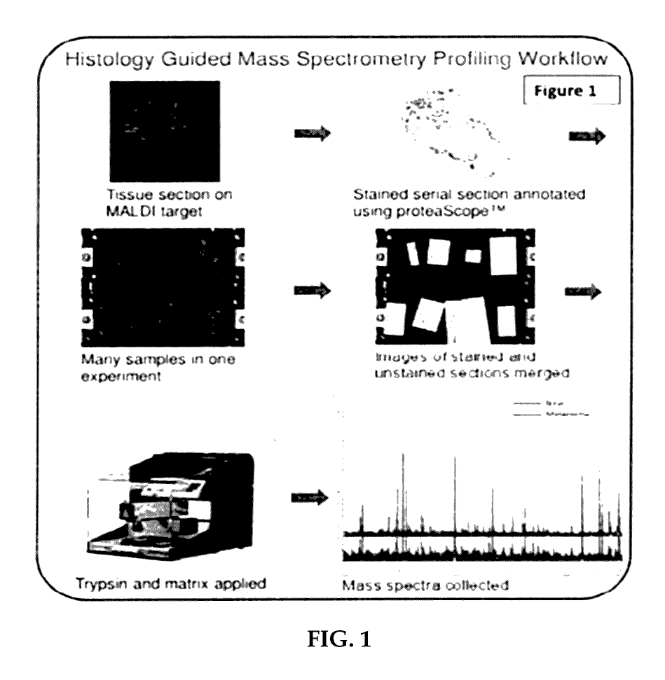

Serial sections, 5 μm thick, were cut from formalin-fixed, paraffin-embedded (FFPE) tissue blocks. One section per sample was mounted onto a conductive glass slide, whereas the consecutive serial section was mounted onto a regular glass slide and stained with hematoxylin and eosin, which served as a reference section (see FIG. 1). Unstained sections were subjected to paraffin removal with xylene and graded ethanol washes. After air-drying, antigen retrieval was performed by heating the sections in Tris buffer. Antigen-retrieved sections were stored in a desiccator at room temperature (about 22° C. to about 25° C.) until matrix deposition for no longer than 2 days.

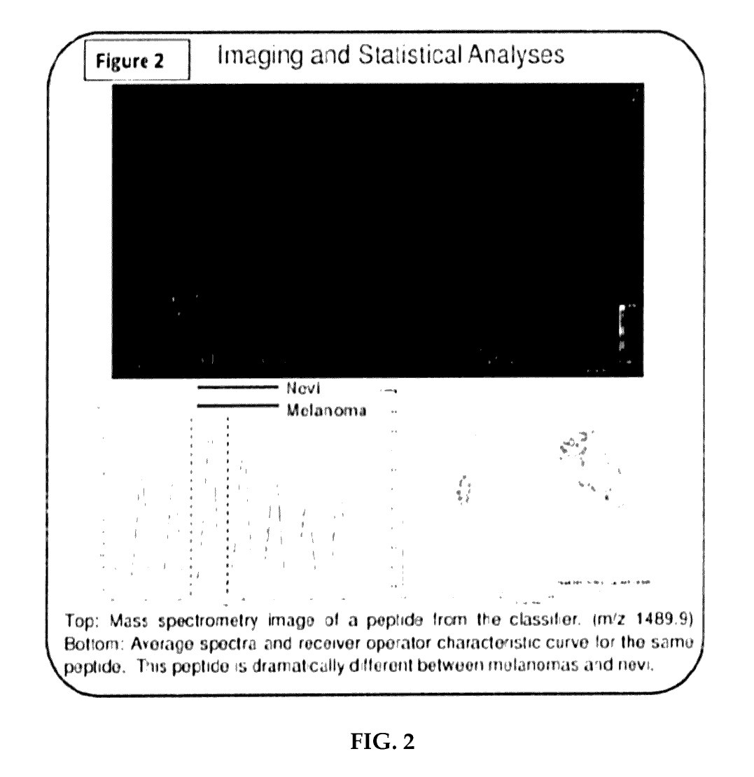

Mass spectral profiles were acquired from the tissue using a histology guided profiling approach as follows. Digital images of the histology slide were acquired using an Olympus VS120 microscope at 20× magnification. The dermatopathologist marked about 100-μm-diameter color-coded areas of interest (about 40 per section) on the digital image of the hematoxylin and eosin stained section. Using image-processing software (e.g., Photoshop), the histology-marked image was merged to an image of the unstained section (FIG. 1). Trypsin was applied to the entire surface of the unstained tissue sections using a SunChrom SunCollect robotic reagent sprayer. The digestion was allowed to proceed in a humidified environment for about 4 hours at about 37° C. An α-Cyano-4-hydroxycinnamic acid matrix was applied via the SunCollect robotic sprayer. The merged annotated images were loaded into the mass spectrometer (ultrafleXtreme, Bruker Daltonics) and after alignment using fiducial points, mass spectra were collected only from the annotated areas (FIG. 2).

Development of Training Models.

The mass spectra profiles were exported and statistical analyses and classification algorithm generation was carried out using: ClinProTools (Bruker) for the genetic algorithm or SCiLS Lab 2016b for the linear discriminant analysis. For the analysis, samples were sorted into training, validation, and classification sets.

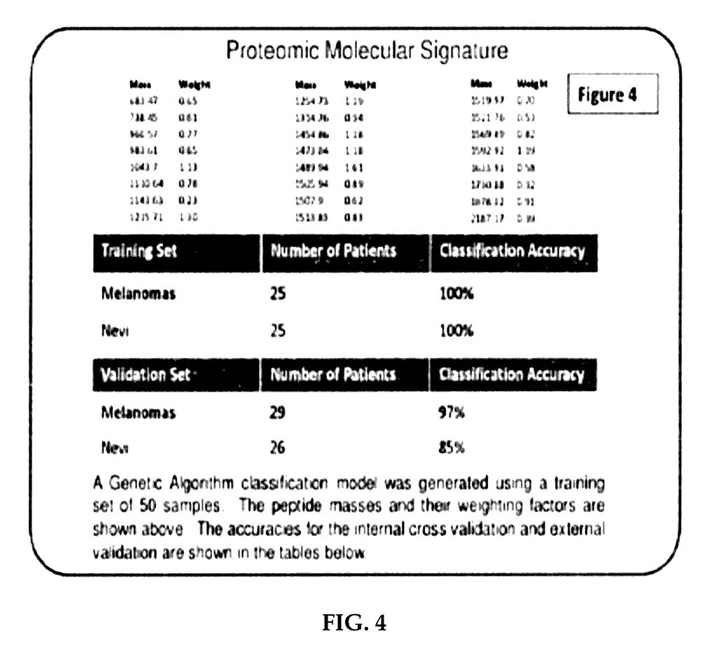

Statistical analysis of the mass spectrometry data using the genetic algorithm allowed for objective generation of a statistical model. The genetic algorithm comprised 24 peaks, determined by statistical comparison of the peaks in the training set for benign melanocytic nevi and malignant melanomas. The 24 peaks represent different peptides, which are differentially expressed in both groups. These peptides have the following masses: 683.47, 738.45, 960.57, 983.61, 1043.7, 1110.64, 1143.63, 1215.71, 1254.73, 1314.76, 1454.86, 1473.84, 1489.94, 1505.94, 1507.9, 1513.83, 1519.97, 1521.76, 1569.89, 1592.92, 1633.91, 1730.88, 1878.02, 2187.17, (FIG. 4). After these 24 peaks were established from the training set, the cases from the validation cohort were subjected to the algorithm.

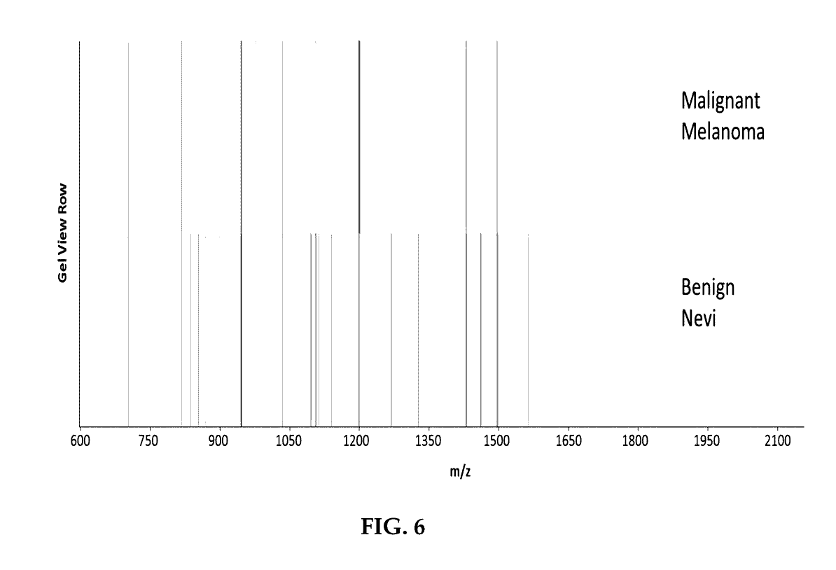

Alternatively, or in conjunction with the genetic algorithm generation, hypothesis testing and discriminative m/z value receiver operating characteristics (ROC) analysis was carried out on the training set (see FIG. 6) using a linear discriminant analysis. In this case, over 166 peaks from the full spectral analysis were identified which were significant with Bonferroni correction, 92 of these peaks had areas under the receiver operator characteristics curve >0.8 or <0.2.

After development of a genetic algorithm model or linear discriminant analysis model on the training set, it was applied to an independent validation set.

Patient-Level Characteristics.

The cohort of benign melanocytic nevi (BMN) came from 51 patients, who ranged from 10 to 62 years of age (mean-25), 29 female and 22 male patients. The nevi were distributed on the trunk (31), head and neck (12), upper extremity (6), and lower extremity (2). None of the lesions recurred or metastasized, and all patients are alive with a follow-up ranging from 4 to 11 years (mean, 6.3). The melanoma (MM) cohort comprised 54 patients from 26 to 97 years old (mean, 68), 21 female and 33 male patients. The distribution was as follows: trunk (22), head and neck (12), upper extremity (10), and lower extremity (10). The depth of the MM ranged from 1.0 to 16.0 mm (mean, 4.8 mm). The follow-up ranged from 4 to 24 years (mean, 13).

Results.

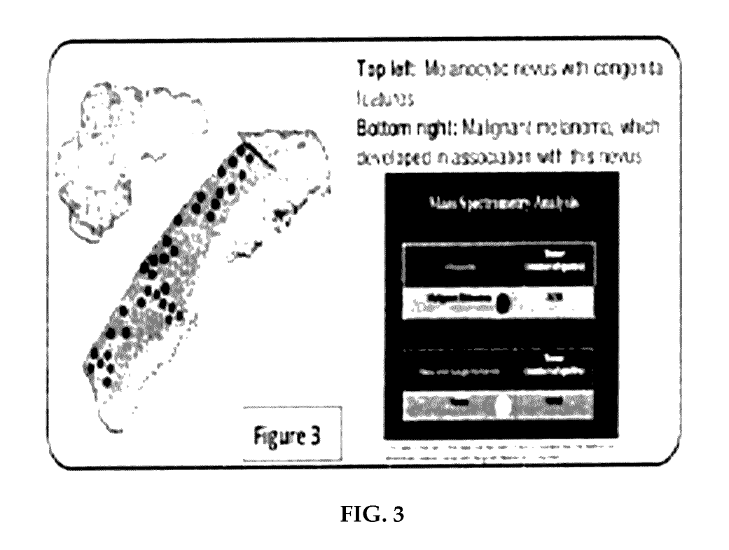

Mass spectra from each dotted area (FIG. 3) on each sample from the training set were obtained. Data were analyzed using ClinProTools to develop a GA, or SCiLS Lab 2016b for the linear discriminant analysis, as described above, and classification models were built using a training set of biopsies from 25 BMN and 25 MM. After a molecular signature was determined based on data from the training set, it was then tested on a validation cohort.

For the genetic algorithm, the validation cohort was 26 BMN and 29 MM. The method correctly recognized 22 of 26 BMN (85%) and 28 out of 29 MM (97%) in the validation cohort (FIG. 4). Thus, the mass spectrometry imaging method using a genetic algorithm showed a sensitivity of about 97% and a specificity of about 85% in correctly identifying MM based on tumor analysis in the validation set.

For the linear discriminant analysis, the validation cohort was 25 BMN and 29 MM. The method correctly recognized 25 of the 25 BMN (100%) and 28 of the 29 MM (97%). Thus, the mass spectrometry imaging method using a linear discriminant analysis showed a sensitivity of 100% and a specificity of about 97% in correctly identifying MM based on tumor analysis in the validation set.

Molecular imaging and signature identification by mass spectrometry imaging may allow clinicians to look beyond classic histology. Gene expression may be useful for distinguishing melanocytic nevi from malignant melanomas. However, it does not always correlate with protein translation and does not account for post-translational modification. Since both protein expression level and post-translational modification state have fundamental effects on cellular function or dysfunction, it may be more meaningful to analyze proteins and peptides that are involved in the development and progression of a cancer. The parent proteins of the peptides that form the classification algorithm may be identified and serve as targets for treatment or for other applications such as immunohistochemical staining. A unique proteomic signature may be obtained for each disease that is studied by this approach. Mass spectrometry imaging (MSI) has the ability to discover molecular signatures of cancer and diseases typically comprised of 5-25 different proteins that together result in robust diagnostic patterns.

In various embodiments, mass spectrometry imaging may be used to study benign melanocytic nevi and malignant melanomas in search of proteomic differences.

Further, proteomic signatures established using MSI classification may be used as a supplement to standard histology in evaluation of melanocytic lesions.

Mass spectrometry imaging and analysis may be useful in cases that are histologically equivocal, and a firm diagnosis of benign melanocytic nevus or malignant melanoma cannot be made with absolute certainty.

Mass spectrometry imaging may provide one or more of the following advantages: mass spectrometry works in formalin-fixed paraffin embedded tissue sections; mass spectrometry is a reliable method to differentiate benign nevi from malignant melanoma based on proteomic differences; mass spectrometry can help in the diagnosis of ambiguous cases by histology; and mass spectrometry is objective, fast, and relatively inexpensive.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to the systems and methods described herein.

What is claimed is:

1. A method for differentiating benign melanocytic nevi from malignant melanomas, the method comprising:
(a) subjecting a skin lesion sample from a patient to mass spectrometry;
(b) obtaining a mass spectrometry profile from the skin lesion sample;
(c) comparing the mass spectrometry profile of the skin lesion sample to a reference profile, wherein the reference profile includes a statistical average profile from a plurality of known normal, benign melanocytic nevi, and/or a plurality of known malignant melanoma; and
(d) classifying the skin lesion sample as a benign melanocytic nevus or a melanocytic malignant melanoma based on a level of similarity between the mass spectrometry profile of the skin lesion sample and the reference profile.

2. The method of claim 1, wherein the statistical average profile is generated using a genetic algorithm which defines one or more markers.

3. The method of claim 1, wherein the statistical average profile is generated using a genetic algorithm which defines six or more markers.

4. The method of claim 2, wherein the statistical average profile comprises markers represented by peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143.63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2.

5. The method of claim 1, wherein the statistical average profile is generated using a linear determinant analysis on a total spectrum from the plurality of known normal, benign melanocytic nevi, and/or the plurality of known malignant melanoma.

6. The method of claim 5, wherein the linear discriminant analysis shows a sensitivity and a specificity of at least 95% in correctly classifying the skin lesion sample.

7. The method of claim 1, wherein the method has a sensitivity and a specificity of at least 95% in correctly classifying the skin lesion sample.

8. The method of claim 1, wherein the mass spectrometry is matrix assisted laser desorption mass spectrometry.

9. The method of claim 1, comprising repeating the steps of (a)-(d) on the skin lesion sample.

10. The method of claim 1, wherein the skin lesion sample comprises melanocytic components.

11. The method of claim 1, wherein the skin lesion sample comprises stromal components and/or benign melanocytic nevi components.

12. The method of claim 1, wherein both melanocytic and stromal components of the skin lesion sample are subjected to mass spectrometry.

13. The method of claim 1, comprising treating the patient with at least one of chemotherapy, immunotherapy, toxin therapy, surgery, and radiotherapy when the patient is identified as having malignant melanoma.

14. The method of claim 1, comprising performing histologic analysis on the skin lesion sample.

15. The method of claim 1, wherein the plurality of known normal, benign melanocytic nevi, and the plurality of known malignant melanoma are classified by immunohistochemical analysis, genetic analysis, patient outcome, or a combination thereof.

16. The method of claim 1, comprising immunohistochemical analysis on the skin lesion sample.

17. A method for differentiating benign melanocytic nevi from malignant melanomas, the method comprising:
(a) subjecting a skin lesion sample from a patient to mass spectrometry;
(b) obtaining a mass spectrometry profile from the skin lesion sample;
(c) comparing the mass spectrometry profile of the skin lesion sample to a reference profile, wherein the reference profile includes a statistical average profile from a plurality of known normal, benign melanocytic nevi, and/or a plurality of known malignant melanoma; and
(d) classifying the skin lesion sample as a benign melanocytic nevus or a malignant melanoma based on a level of similarity between the mass spectrometry profile of the skin lesion sample and the reference profile,
wherein the statistical average profile is generated using a linear determinant analysis on a total spectrum from the plurality of known normal, benign melanocytic nevi, and/or the plurality of known malignant melanoma, and
wherein the linear discriminant analysis shows a sensitivity and a specificity of at least 95% in correctly classifying the skin lesion sample.

18. The method of claim 17, wherein the sensitivity and specificity is at least 97% in correctly classifying the skin lesion sample.

19. A method for differentiating benign melanocytic nevi from malignant melanomas, the method comprising:
(a) subjecting a skin lesion sample from a patient to mass spectrometry;
(b) obtaining a mass spectrometry profile from the skin lesion sample;
(c) comparing the mass spectrometry profile of the skin lesion sample to a reference profile, wherein the reference profile includes a statistical average profile from a plurality of known normal, benign melanocytic nevi, and/or a plurality of known malignant melanoma; and
(d) classifying the skin lesion sample as a benign melanocytic nevus or a melanocytic malignant melanoma based on a level of similarity between the mass spectrometry profile of the skin lesion sample and the reference profile,
wherein the statistical average profile is generated using a genetic algorithm which defines one or more markers.

20. The method of claim 19, the statistical average profile comprises markers represented by peptide peaks at one or more of m/z 683.47±0.2, m/z 738.45±0.2, m/z 960.57±0.2, m/z 983.61±0.2, m/z 1043.70±0.2, m/z 1110.64±0.2, m/z 1143.63±0.2, m/z 1215.71±0.2, m/z 1254.73±0.2, m/z 1314.76±0.2, m/z 1454.86±0.2, m/z 1473.84±0.2, m/z 1489.94±0.2, m/z 1505.94±0.2, m/z 1507.90±0.2, m/z 1513.83±0.2, m/z 1519.97±0.2, m/z 1521.76±0.2, m/z 1569.89±0.2, m/z 1592.92±0.2, m/z 1633.91±0.2, m/z 1730.88±0.2, m/z 1878.02±0.2, and m/z 2187.17±0.2.
